(19) United States
(12) Patent Application Publication
(10) Pub. No.: US 2009/0048660 A1
(43) Pub. Date: Feb. 19, 2009

(54) IMPLANT OF A BIOCORRODABLE MAGNESIUM ALLOY AND HAVING A COATING OF A BIOCORRODABLE POLYPHOSPHAZENE

(75) Inventor: Nina Adden, Nuernberg (DE)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 12/192,729

(22) Filed: Aug. 15, 2008

(30) Foreign Application Priority Data
Aug. 17, 2007 (DE) 10 2007 038 799.9

(51) Int. Cl. A61F 2/06; A61F 2/02

(52) U.S. Cl. 623/1.15; 623/11.11; 623/1.49

(57) ABSTRACT

An implant of a biocorrodable metallic material comprising a coating having a biocorrodable polyphosphazene.

### PRIORITY CLAIM

This patent application claims priority to German Patent Application No. 10 2007 038 799.9, filed Aug. 17, 2007, the disclosure of which is incorporated herein by reference in its entirety.

### FIELD

The present disclosure relates to an implant of a biocorrodable magnesium alloy having a coating.

### BACKGROUND

Implants in a variety of embodiments have gained acceptance in modern medical technology. For example, implants are used to support blood vessels, hollow organs and duct systems (endovascular implants) for fastening and temporarily securing tissue implants and tissue transplants. Implants are also used for orthopedic purposes, e.g., as nails, plates or screws.

Implantation of stents has become established as one of the most effective therapeutic measures in treatment of vascular diseases. The purpose of stents is to assume a supporting function in the hollow organs of a patient. Stents of a traditional design, therefore, have a filigree supporting structure comprised of metallic struts which are initially in a compressed form for introduction into the body of the patient and then are widened at the site of application. One of the main areas of application of such stents is for permanently or temporarily widening vascular occlusions and keeping the occlusions open, in particular, constrictions (stenoses) of the myocardial vessels. In addition, aneurysm stents which serve to support damaged vascular walls are also known.

Stents have a circumferential wall of a sufficient supporting strength to keep the constricted vessel open to the desired extent and have a tubular base body through which blood continues to flow unhindered. As a rule, the supporting vascular wall is formed by a mesh-like supporting structure which allows the stent to be inserted in a compressed state with a small outside diameter as far as the stenosed site to be treated in the respective vessel and to widen the vessel at the stenosed site, e.g., with the help of a balloon catheter, so that the vessel has the desired enlarged inside diameter. To avoid unnecessary vascular damage, there should not be any elastic recoil of the stent or the elastic recoil should only be of a minor extent after widening and after removal of the balloon, so that the stent need only be widened slightly beyond the desired final diameter when the stent is widened. Additional criteria which are desirable with respect to a stent include, for example, a uniform surface coverage and a structure that allows a certain flexibility with respect to the longitudinal axis of the stent. In practice, the stent is usually made of a shaped metal material in order to achieve the mechanical properties mentioned hereinabove.

In addition to the mechanical properties of a stent, the stent should be made of a biocompatible material to prevent rejection reactions. Stents are currently used in approximately 70% of all percutaneous interventions, but an in-stent restenosis occurs in 25% of all cases due to excessive neointimal growth which is induced by a great proliferation of the arterial smooth muscle cells and a chronic inflammation reaction. Various approaches have been pursued to solve the problem of lowering the rates of restenosis.

According to one approach for reducing the incidence of restenosis, an active pharmaceutical substance (active ingredient) which counteracts the mechanisms of restenosis and supports the progress of healing is provided on the stent. The active ingredient is applied in pure form as a coating or embedded in a carrier matrix or is packed into cavities of the implant. Examples include the active ingredients sirolimus and paclitaxel.

Another currently promising approach to solving the problem lies in the use of biocorrodable metals and their alloys because a permanent supporting function of the stent is not usually necessary. Although initially damaged, the body tissue regenerates. For example, German Patent Application No. 197 31 021 A1 proposes that medical implants should be shaped from a metallic material whose main ingredient is iron, zinc or aluminum and/or an element from the group of alkali metals or alkaline earth metals. Alloys based on magnesium, iron and zinc are described as being especially suitable. Secondary ingredients of the alloys may include manganese, cobalt, nickel, chromium, copper, cadmium, lead, tin, thorium, zirconium, silver, gold, palladium, platinum, silicon, calcium, lithium, aluminum, zinc and iron. In addition, German Patent Application No. 102 53 634 describes the use of a biocorrodable magnesium alloy with a magnesium content of >90%, yttrium 3.7-5.5%, rare earth metals 1.5-4.4% and remainder <1%. These are suitable, in particular, for producing an endoprothesis, e.g., in the form of a self-expanding or balloon-expandable stent. The use of biocorrodable metallic materials in implants could lead to a definite reduction in rejection reactions or inflammation reactions.

The combination of active ingredient release and biocorrodable metallic material seems to be especially rich in prospects. The active ingredient is applied as a coating or is introduced into a cavity in the implant, usually embedded in a carrier matrix. For example, stents of a biocorrodable magnesium alloy with a coating of a poly(L-lactide) are known in the art. However, it has been found that the degradation of known polymer coatings on stents made of a biocorrodable magnesium alloy is accelerated. This may be attributed to, among other things, strongly basic conditions which are established as a result of the degradation of the magnesium alloy. Furthermore, the products of degradation of the polymer coating, which are often acidic, can lead to an inflammatory reaction of the surrounding tissue, i.e., the material shows only a moderate biocompatibility.

### SUMMARY

The present disclosure describes several exemplary embodiments of the present invention.

One aspect of the present disclosure provides an implant of a biocorrodable magnesium alloy comprising a coating of biocorrodable polyphosphazene.

Another aspect of the present disclosure provides a method of using biocorrodable polyphosphazenes as a coating material for a stent made of a biocorrodable metallic alloy.

### DETAILED DESCRIPTION

A first aspect of the present disclosure provides an implant made of a biocorrodable magnesium alloy and having a coating comprising or containing a biocorrodable polyphosphazene.

Polyphosphazenes are polymers with the general structure of formula (1)

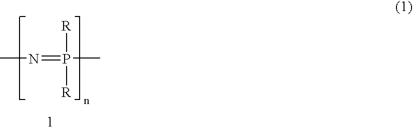

having a polymer backbone which is alternately constructed of phosphorus atoms and nitrogen atoms. The two remaining bonds on the phosphorus correspond to the substituent R.

In the case of biocorrodable polyphosphazenes, R preferably stands for a substituent formed by coupling to a primary or secondary amine or an amino acid ester. To control the degradation rate, R may also denote an alkoxy group in addition to the aforementioned substituents. The aforementioned polyphosphazenes are preferably produced by reaction of polydichlorophosphazenes with the desired amine or amino acid ester (like or according to Adv. Drug Del. Rev. 2003, 55, 467; Biotech. Bioeng. 1996, 52, 102; or J Biomed Mater Res 2007, 80A, 661).

According to an exemplary embodiment, R is a substituent formed by coupling to an α-amino acid ester of general formula (2)

H₂NCHR′COOR″ (2)

where R′ stands for a canonical or non-canonical radical of a proteinogenic amino acid. R″ is an alkyl radical with 1-10 carbon atoms, preferably methyl or ethyl. In this way the degradation rate of the polymer can be influenced easily and degradation of the polymer leads to products that are identical to the natural products and have very little or no adverse effects. Larger and more hydrophobic groups R′ and R″ lead to slower degradation of the polyphosphazene. R is especially preferably a substituent formed by coupling to a metal ester or an ethyl ester of the amino acids glycine, alanine, valine or phenylalanine.

The degradation rate may also be reduced by replacing the substituent R to a slight extent by a moderately corrodable or nonbiocorrodable substituent, e.g., by reacting the precursor polydichlorophosphazene with small amounts of methylamine or ethanol.

The polyphosphazene has a molecular weight between 10,000 g/mol and 10,000,000 g/mol, preferably between 100,000 g/mol and 5,000,000 g/mol.

The degradation rate of the polyphosphazene is between 3 days and 600 days, preferably between 20 days and 360 days. This is not affected by the basic conditions which occur due to degradation of the degradable metallic material.

A coating according to the present disclosure is an application of the components to the base body of the implant, in particular, stents, in at least some sections. The entire surface of the base body of the implant/stent is preferably covered by the coating. A layer thickness is preferably in the range from 1 nm to 100 μm, especially preferably 300 nm to 20 μm. The coating is formed from or contains a biocorrodable polyphosphazene. The amount by weight of polyphosphazene in the components of the coating forming the carrier matrix amounts to at least 30%, preferably at least 50%, especially preferably at least 70%. A blend of different polyphosphazenes may be used. The components of the coating comprise the materials that act as a carrier matrix, i.e., materials which are necessary for the functional properties of the carrier matrix, e.g., including additives to improve the viscosity properties, gelation and proccessability. These components do not include the active ingredients or marker materials that are optionally added. The coating is applied directly to the implant surface or an adhesive layer is applied first. These may be, for example, silanes or phosphonates that have a reactive terminal group (COOH, OH, NH₂, aldehyde) or an oxidic conversion layer of the base material and are applied to the surface of the base material.

The polyphosphazenes used according to the present disclosure are highly biocompatible and biocorrodable. The processing may be performed according to standard coating methods. Single-layer or multilayer systems may be created (e.g., so-called base coat, drug coat or topcoat layers).

The polyphosphazene may act as a carrier matrix for pharmaceutical active ingredients, in particular, anti-proliferative active ingredients such as sirolimus, everolimus, biolimus and paclitaxel and anti-inflammatory active ingredients such as pimecrolimus, and/or for marker materials such as X-ray markers, preferably tungsten carbide or finely dispersed gold and/or magnetic resonance markers. The X-ray marker cannot be applied directly to the product in the case of implants made of a biocorrodable metallic material because the marker would influence the degradation of the stent by forming local elements. However, in the matrix of polyphosphazenes, the marker is shielded from the base body.

For purposes of the present disclosure, the term biocorrodable refers to metallic or polymeric materials in which degradation takes place in a physiological environment ultimately resulting in loss of mechanical integrity of the entire implant or the part of the implant formed from this material.

For purposes of the present disclosure, the term biocorrodable magnesium alloy refers to a metallic structure whose main component is magnesium. The main component is the alloy component whose amount by weight in the alloy is the greatest. An amount of main component is preferably more than 50 wt %, in particular, more than 70 wt %. The biocorrodable magnesium alloy preferably contains yttrium and other rare earth metals because such an alloy is characterized by its physicochemical properties and high biocompatibility and, in particular, also its degradation products. A magnesium alloy with the composition 5.2-9.9 wt % rare earth metals, including 3.7-5.5 wt % yttrium and the remainder <1 wt %, is especially preferred, where magnesium constitutes the remaining portion of the alloy to a total of 100 wt %. This magnesium alloy has already confirmed its special suitability in experiments and in preliminary clinical trials, i.e., the magnesium alloy has manifested a high biocompatibility, favorable processing properties, good mechanical characteristics and an adequate corrosion behavior for the intended purpose. For purposes of the present disclosure, the general term “rare earth metals” includes scandium (21), yttrium (39), lanthanum (57) and the fourteen elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71).

The composition of polyphosphazene and the magnesium alloy are to be selected so that they are biocorrodable. Artificial plasma (composition NaCl 6.8 g/L, CaCl₂ 0.2 g/L, KCl 0.4 g/L, MgSO₄ 0.1 g/L, NaHCO₃ 2.2 g/L, Na₂HPO₄ 0.126 g/L, NaH₂PO₄ 0.026 g/L) as specified for biocorrosion tests according to EN ISO 10993-15:2000 is used as the test medium for testing the corrosion behavior of polymeric materials or alloys. A sample of the material to be tested is stored with a defined amount of the test medium at 37° C. in a sealed sample container. At intervals of time, based on the corrosion behavior to be expected, from a few hours up to several months, the samples are removed and tested for traces of corrosion by methods know in the art. The artificial plasma according to EN ISO 10993-15:2000 corresponds to a medium resembling blood and thus represents an opportunity to reproducibly simulate a physiological environment.

For purposes of the present disclosure, implants refer to devices introduced into the body by a surgical procedure and comprise fastening elements for bones, e.g., screws, plates or nails, surgical suture material, intestinal clamps, vascular clips, prostheses in the area of hard and soft tissue and anchoring elements for electrodes, in particular, pacemakers or defibrillators.

The implant is preferably a stent. Stents of a traditional design have a filigree supporting structure comprised of metallic struts which are present initially in an unexpanded state for introduction into the body and which are then widened at the site of application into an expanded state. On the basis of the type of use, brittle coating systems are not suitable; however, polyphosphazenes have especially suitable material properties, such as an adequate viscosity and flexibility, for these purposes. The stent may be coated before or after crimping onto a balloon.

A second aspect of the present disclosure relates to the use of biocorrodable polyphosphazenes as the coating material for a stent made of a biocorrodable magnesium alloy.

The present invention is explained in greater detail below on the basis of an exemplary embodiment.

### EXAMPLES

### Example 1

### Coating Absorbable Metal Stents

A stent made of the biocorrodable magnesium alloy WE43 (97 wt % magnesium, 4 wt % yttrium, 3 wt % rare earth metals, not including yttrium) is coated as described below.

A solution of a polyphosphazene in tetrahydrofuran is prepared (30 wt %). The polyphosphazene used has phenylalanine ethyl ester and ethyl glycinate side groups in a ratio of 1.4:0.6. This synthesis has been described by Carampin et al. (J Biomed Mater Res, 2007, 80A, 661). A pharmaceutical drug may be added to this solution as needed.

The stent is cleaned to remove dust and residues and is clamped in a suitable stent coating apparatus (DES Coater, internal development by the Biotronik company). With the help of an airbrush system (EFD company or spraying system), the rotating stent is coated on one side under constant ambient conditions (room temperature; 42% atmospheric humidity). At a nozzle distance of 20 mm, a stent 18 mm long is coated after approximately ten minutes. After reaching the intended layer weight, the stent is dried for five minutes at RT before the uncoated side is coated in the same way after rotating the stent and clamping it again. The finished coated stent is dried in a vacuum oven (Vakucell; company MMM) for 36 hours at 40° C.

A layer thickness of the applied polyphosphazene is approximately 15 μm.

### Example 2

### Coating Absorbable Metal Stents

A stent made of the biocorrodable magnesium alloy WE43 (97 wt % magnesium, 4 wt % yttrium, 3 wt % rare earth metals, not including yttrium) is coated as described below.

A solution of a polyphosphazene in chloroform is prepared (5 wt %). The polyphosphazene used has phenylalanine ethyl ester and glycine ethyl ester side groups in a 0.5:1.5 ratio. This synthesis has been described by Carampin et al. (J Biomed Mater Res, 2007, 80A, 661). A pharmaceutical drug may be added to this solution as needed.

The stent is cleaned to remove dust and residues and is attached to a hook. With the help of a dipping system (Specialty Coating Systems), the stent is immersed in the solution under constant ambient conditions (room temperature; 42% atmospheric humidity) and pulled out again at a rate of 1 mm per minute. The stent is dried at RT for five minutes: several immersion passes are possible. The finished coated stent is dried in a vacuum oven (Vakucell; company MMM) for 36 hours at 40° C.

A layer thickness of the applied polyphosphazene is approximately 20 μm.

All patents, patent applications and publications referred to herein are incorporated by reference in their entirety.

What is claimed is:

1. An implant, comprising: a biocorrodable magnesium alloy having a coating of biocorrodable polyphosphazene.

2. The implant of claim 1, wherein the implant is a stent.

3. The implant of claim 1, wherein the coating contains either an active ingredient or a marker material.

4. The implant of claim 1, wherein the polyphosphazene is a polymer of formula (1) 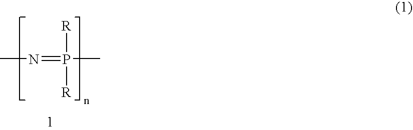
where R stands for a substituent which is formed by coupling to either a primary or secondary amine or an amino acid ester.

5. The implant of claim 4, wherein R is a substituent formed by coupling to an α-amino acid ester of general formula (2)
H₂NCHR′COOR″ (2)
wherein R′ stands for either a canonical or non-canonical radical of a proteinogenic amino acid and R″ is an alkyl radical with 1-10 carbon atoms.

6. The implant of claim 5, wherein R is a substituent formed by coupling with a either methyl ester or ethyl ester of the amino acids selected from the group consisting of glycine, alanine, valine and phenylalanine.

7. A method of forming a biocorrodable stent, comprising:
a) providing a stent made of a biocorrodable metallic alloy; and
b) coating the alloy with a material comprising a biocorrodable polyphosphazene.
